(19) United States
(12) Patent Application Publication
(10) Pub. No.: US 2025/0040866 A1
(43) Pub. Date: Feb. 6, 2025

(54) SYSTEM AND COMPUTER METHOD INCLUDING A DIGITAL BIOMARKER (DBM), CLART, FOR REMOTELY ASSESSING HEPATIC ENCEPHALOPATHY FOR CIRROHTIC PATIENTS

(71) Applicant: CYBERLIVER LIMITED, Stanmore (GB)

(72) Inventors: Kalkivayi Seshagiri RAVI KUMAR, Manchester (GB); Rajeshwar Prosad MOOKERJEE, Northwood (GB)

(21) Appl. No.: 18/717,193

(22) PCT Filed: Dec. 7, 2022
(86) PCT No.: PCT/IB2022/061907

§ 371 Date: Jun. 6, 2024

Related U.S. Application Data
(60) Provisional application No. 63/286,686, filed on Dec. 7, 2021.

(51) Int. Cl. A61B 5/00; G16H 50/20

(52) U.S. Cl. CPC A61B 5/4076; A61B 5/4244; A61B 5/7435; G16H 50/20

(57) ABSTRACT

The invention concerns a system for assessing hepatic encephalopathy of at least one patient P, comprising: —at least one patient application module (PM) to take at least one animal recognition test for assessing hepatic encephalopathy, and to determine test results; —at least one interpretation module (IM) receiving the test results, and configured to interpret the test results in order to determine parameters of hepatic encephalopathy such as a risk thereof, —at least one practitioner application module (DM) receiving the parameters of hepatic encephalopathy to provide a diagnosis aid to a practitioner (D). According to the invention, the animal recognition test is based on animal images (TI) and corresponding text descriptions (TT) from a junior school repertoire, for easy and rapid tests; and/or the animal recognition test is made without colour shades parameters, for colour blind patients. The invention further provides a simple interpretation of the test results.

### FIELD OF THE INVENTION

The present invention relates to a system and computer implemented method for conditions related to decompensated cirrhosis. The invention more generally relates to the assessment of neuropsychiatric abnormalities in patients with liver dysfunction, in particular the assessment of hepatic encephalopathy (HE); as well as enabling delivery of interventions—digital therapies—in this regard.

Hepatic encephalopathy (HE) is a syndrome usually observed in patients with severely impaired liver function, most commonly seen in advanced cirrhosis. Hepatic encephalopathy is defined as a spectrum of neuropsychiatric abnormalities in patients with liver dysfunction, after exclusion of brain disease. Patients with decompensated cirrhosis are at high risk of hepatic encephalopathy (HE) and need to be constantly monitored for their higher cognitive function. The development of HE has implications on quality of life, overall survival, as well as an increase in risks of falls and accidents, and thus has a large health economic burden.

### BACKGROUND OF THE INVENTION

Some solutions of the prior art synthesize and define a sequential framework of core principles for selecting and developing measurements in research and clinical care that are meaningful for patients.

Another prior art solution uses paper-and-pencil tests that make up a Psychometric Hepatic Encephalopathy Score (PHES) assessing attention, visual perception and spatial awareness abilities.

Other prior art solutions are provided, such as one that uses what is called the EncephalApp, which is a colour/word based form of stroop test, for determining onset of HE. The drawback of the application is that it uses a colour-based test, which cannot be assessed in patients who have colour blindness or vision issues. The test is also time-bound and complex to explain and reproduce.

In essence, many solutions of the prior art involve long and tedious procedures, and need significant amounts of materials and time for a practitioner.

Other solutions are not easy to implement to colour-blind patients, and/or are complex to explain and to be implemented by patients.

### SUMMARY OF THE INVENTION

The objective of the invention is to overcome the problems of the prior art, in particular to provide a solution that is easy to handle and to implement, preferably at least partly from home; and to provide a solution that is quick to explain and easy to implement to a large number of people, including colour blind people.

In order to meet this objective, the invention concerns a system for assessing hepatic encephalopathy of at least one patient, the system comprising:
at least one patient application module configured to be used to take at least one animal recognition test for assessing hepatic encephalopathy, and to determine test results;
at least one interpretation module receiving the test results, and configured to interpret the test results in order to determine parameters of hepatic encephalopathy,
at least one practitioner application module configured to receive at least the parameters of hepatic encephalopathy to provide a diagnosis aid.

According to a first aspect of the invention, the animal recognition test is based on animal images and corresponding text descriptions from a junior school repertoire; and/or wherein the animal recognition test is made without colour shades parameters.

“Images and text descriptions” from a “a junior school” repertoire may be interpreted as images and text descriptions that are easily understood by a child of a junior school age. A test made “without colour shades” parameters may be interpreted as a test in which a clear distinction and understanding of different colours and/or different colour shades is not a parameter which intervene in the understanding and/or the completion of the test. Contrary to the prior art in which some stroop tests are based on a recognition of a word of a given colour with letters of a different given colours. The CLART test of the invention does not incudes such colour parameters, which makes it easy to take for colour blind patients. The CLART test is mainly based on the recognitions of forms of the animals and recognition of the corresponding name in the preferred embodiment.

A second aspect of the invention relates to the interpretation of the test results. The test results may include one or more of a score, a success or failure to the test. According to an embodiment, the test results comprise at least:
a time to perform the test compared to a time baseline or median or mean;
a number of failed or successfully completed tests, and
a number of test attempts.

This enables to limit the calculations of the algorithms and to spare the corresponding calculation time and energy.

According to an embodiment, the test results comprise a score scaling a combination of the time to perform, the number of failed or successfully completed tests, and the number of attempts. This enables to have a quick interpretation in the diagnosis aid, based on three data points of the test results.

The parameters of hepatic encephalopathy may include a positive/negative diagnosis, or a positive/negative probability.

According to an embodiment, the parameters of hepatic encephalopathy comprise a high, low or medium risk:
“low” meaning “low probability of developing HE event”;
“medium” meaning a “high probability of developing HE event”;
“High” meaning “detection of HE event at the time of patient taking the test”.

This enables to provide a quick and concise aid to the clinicians, helping them to decide the next management steps regarding for example the medication and/or need for hospitalisation.

Advantageously, the present invention enables high-quality patient engagement using digital means. In addition, the invention is an improvement over the paper-and-pencil test by encapsulating the different cognitive modalities from the above in a rapid, easy to perform, digital context, from the convenience of the patient's home.

Moreover, the invention uses an animal recognition test (i.e. CLART test) which can work for patients with limited education and/or colour blindness and visual issues, and is simple and quick to perform.

According to an embodiment, the animal recognition test is made by typing the right animal image or the right text description on a tactile screen.

This enables to simplify the CLART test and have it quickly performed to improve the engagement of the patients.

According to an embodiment, the system further comprises a prescription module to prescribe at least one new animal recognition test to be performed through the patient module of a patient.

This enables to adapt the following up of specific patients with specific sets of CLART tests at specific frequencies, hours, etc.

According to an embodiment, the system further comprises a reminder module configured to signal or remind the patient that a new animal recognition test is to be performed.

This enables to avoid missing of important CLART tests for the following up of the patient.

According to an embodiment, the system further comprises at least one patient device comprising a corresponding patient module; at least one practitioner device comprising a corresponding practitioner module; and at least one server comprising the interpretation module.

This enables to take the CLART tests from home, and to be diagnosed by the practitioners from their office.

The interpretation module may be part of the patient device, practitioner device, and/or server. As part of the server, this enables to lower the calculation and processing time and energy of the patient device, and of the practitioner device.

The devices may be selected from the group comprising mobile devices such as smartphones, tablets, laptop computers; or desktop devices such as PCs, tactile boards. Preferably, the patient device is a smartphone to enable to take multiple CLART tests without inconvenience; and the practitioner device is a desktop or laptop computer to be more convenient for use in a hospital or medical centre, and to view more clearly some details.

The patient device may comprise a tactile screen.

The prescription module is preferably triggered through the practitioner device and/or is included in the practitioner module. This enables to have a practitioner control the prescription and follow up of the patient, which is more regular, sustainable and adaptable.

The invention further relates to a kit for assessing hepatic encephalopathy of at least one patient, the kit comprising parts of the system of the invention. Embodiments of the system listed above may apply to embodiments of the kit.

In particular, the invention further relates to a kit for assessing hepatic encephalopathy of at least one patient, the kit comprising:
at least one patient application for a patient device, wherein the patient application comprises a patient application module configured to be used to take at least one animal recognition test for assessing hepatic encephalopathy, and to determine test results; and/or
at least one practitioner application for a practitioner device, wherein the practitioner application comprises a practitioner application module configured to receive at least parameters of hepatic encephalopathy to provide a diagnosis aid.

In particular, the animal recognition test is based on animal images and corresponding text descriptions from a junior school repertoire; and/or the animal recognition test is made without colour shades parameters.

The kit may be provided to a person having the server or a platform, and the person provides the patient applications to patients and/or practitioner applications to practitioners.

Another aspect of the invention concerns a computer-implemented method for assessing hepatic encephalopathy of at least one patient, based on implementations of the system of the invention.

In particular, another aspect of the invention concerns a computer-implemented method for assessing hepatic encephalopathy of at least one patient, the method comprising:
a step for taking at least one animal recognition test to assess hepatic encephalopathy, and to determine test results;
a step for interpreting to interpret the test results in order to determine parameters of hepatic encephalopathy,
a step for providing to provide a diagnosis aid based on the parameters of hepatic encephalopathy.

According to an embodiment, the animal recognition test is based on animal images and corresponding text descriptions from a junior school repertoire; and/or and wherein the animal recognition test is made without colour shades parameters.

Embodiments of the system listed above may apply to embodiments of the method.

According to an embodiment, the animal recognition test is made by typing the right animal image or the right text description on a tactile screen.

According to an embodiment, the method further comprises a step for prescribing at least one new animal recognition test to be performed by a patient.

According to an embodiment, the method further comprises a step for reminding to signal or remind the patient that a new animal recognition test is to be performed.

According to an embodiment:
the step for taking the at least one animal recognition test is made through at least one patient device;
the step for providing is made through at least one practitioner device; and
the step for interpreting is made through at least one server.

The invention further relates to a computer program product comprising instructions which, when the program is executed by one or more computerised unit, causes the said computerised unit to carry out steps of the method according to the invention.

### BRIEF DESCRIPTION OF THE DRAWINGS

The invention will be further detailed by the description of non-limitative embodiments, and based on the enclosed figures, among which:

FIG. 1 is a general block diagram of a preferred embodiment of the invention; and

FIG. 2 is a general scheme of an animal recognition test (i.e: CLART) being performed in accordance with the invention.

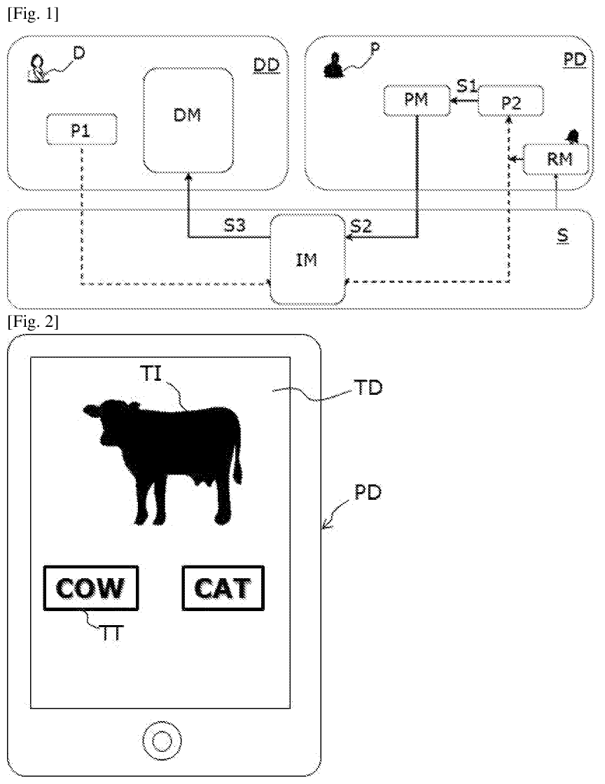

### DETAILED DESCRIPTION OF THE INVENTION

The invention first concerns a system for assessing hepatic encephalopathy of one or more patients P. The system is adapted for multiple patents P, with sets of patients followed up by a specific practitioner D, such as a doctor, professor, or any clinician/practitioner.

The system comprises corresponding patient application modules PM for each patients P. In the preferred embodiment, the application module PM is part of a patient application that may be downloaded and run on a patient device PD, such as a smartphone.

The application module PM is configured to be used to take animal recognition tests (i.e.: CLART test) for assessing hepatic encephalopathy of the patient. Once a CLART test has been performed, the patient application module PM determines test results for in interpretation, such as in the form of a score (also called digital biomarker).

The system further concerns at least one interpretation module IM to interpret the CLART tests and help in assessing hepatic encephalopathy. The interpretation module IM may include algorithms and data issued from researches, in order to interpret the CLART test and data of the patient and assess hepatic encephalopathy.

The interpretation module IM may be implemented in a platform or server S receiving information from the patient, in particular by means of the patient device.

The interpretation module IM receives at least the CLART test results, and may receive also some patient data. Based on its features, the interpretation module IM is configured to interpret the CLART test results in order to determine parameters suggestive of hepatic encephalopathy, to determine need for intervention.

To avoid diagnosis errors, the parameters of hepatic encephalopathy are to be interpreted by the practitioner M.

In this regard, the system further comprises one or more practitioner application module DM to show the parameters of hepatic encephalopathy to the practitioner M. The practitioner application module DM may be implemented in a separate practitioner device MD, such as a computer. The practitioner application module DM receives the parameters of hepatic encephalopathy to provide a diagnosis aid to the practitioner M.

According to the invention, the CLART test is based on animal images TI and corresponding text descriptions TT from a junior school repertoire.

To this end the CLART test may be made by typing the right animal image TI or the right text description TT on a tactile screen, such as shown in [FIG. 2].

Alternatively or in combination, the CLART test is made without colour shades parameters. on a display TD, such as the one of [FIG. 2].

In addition, it is preferred that the CLART tests be prescribed by the practitioner M as an expert. To this end, the system further comprises a prescription module P1, P2 to prescribe at least one new CLART test to be performed through the patient device PD/module PM of the patient. Reminders are preferably provided in the system, based on a reminder module RM, to signal or remind the patient that a new CLART test is to be performed.

The invention also includes a corresponding kit comprising at least the patient application that is provided to one or more patients P.

The invention further relates to the corresponding computer-implemented method including steps performed by the different modules.

The method may include:
a step of taking CLART tests S1;
a step of interpreting to interpret the test results S2, for the parameters of hepatic encephalopathy;
a step of providing S3 to provide a diagnostic aid to the practitioner D.

Another subject-matter of the invention concerns the corresponding computer program product or software or applications to run the claimed system. A specific part may be implemented in each of the devices PD, DD, S, as computerised units.

### Methods and Results

### Method:

Method according to one aspect of the invention, to remotely assess and diagnose Hepatic Encephalopathy (HE) and calculation of CLART, a novel digital biomarker (dBM) to predict the risk of developing HE comprising the following steps:
1. the CLART App (including the patient module PM) is used to capture at least (a) a time taken to complete CLART test, and (b) a number of attempts for completing the full test; (c) the number of failed tests.
2. Algorithms of the interpretation module IM are then used to calculate CLART digital biomarker based on:
a percentage difference from baseline and/or median or mean test completion in time taken to complete the test and cumulative mean/median of completed tests; and
a number of failed attempts compared to the total attempts.

The interpretation module IM then makes a classification of the risk based on the CLART digital biomarker, which preferably includes a score:
based on the CLART score, the possibility of developing Hepatic Encephalopathy is classified as Low, Medium or High: “Low” meaning for example “low probability of developing HE event” for example within the next 72 hours; “Medium” meaning for example a “high probability of developing HE event” for example “within the next 24 hours”; “High” meaning for example “detection of HE event at the time of patient taking the test”.

### Validation:

The method was validated against the clinical data, observations and interventions and outcomes, during the CirrhoCare™ pilot study in decompensated cirrhosis patients.

### Results:

### Case-1: Patient-3, Day-2:

### Data Captured Using the CLART Test:

CLART tests attempts=1 (failed);
CLART-dBM interpretation of HE Risk—Medium.

### Clinical Outcome:

The patient was flagged with medium risk of HE based on the CLART score, derived using the methodology of the invention. The patient was managed at home through early intervention, when on further questioning, fluid imbalance appeared as the driver of new HE.

The result demonstrates that CLART predicted risk is as seen in the real-world clinical setting; but the follow up of the patient was remote and from the comfort of their home.

### Case-2: Patient-3, Day 29:

### Data Captured Using the CLART Test:

CLART tests attempts=2 (completed successfully);
CLART Test completion time=2.6×baselinemean
CLART-dBM interpretation of HE Risk—High.

### Clinical Outcome:

The patient was flagged with high risk of HE based on the CLART score derived using the methodology of the invention. The patient was admitted with HE and a possible skin infection (inflammatory parameters not markedly increased and no positive cultures found) at a local hospital and was treated with empiric antibiotics and fluids during her admission lasting 11 days. She resumed monitoring after return home and had no further events for the rest of the monitoring period.

### Case-3: Patient-9, Day 24:

### Data Captured Using the CLART Test:

CLART tests attempts=4 (3 failed tests, 1 completed successfully);
CLART Test completion time=2.5×baselinemean;
CLART-dBM interpretation of HE Risk=High.

### Clinical Outcome:

The patient was flagged with high risk of HE based on the CLART score derived using the methodology and was admitted into the hospital. On admission, the patient showed no signs of infection or haemorrhage. He was treated with fluids and an increase in laxatives, and Rifaximin was started. His antidiabetic medicine was adjusted. He was discharged after 4 days and continued to do daily measurements after his return home.

### Case-4: Patient-9, Day 35:

### Data Captured Using the CLART Test:

CLART tests attempts=1 (completed successfully);
CLART Test completion time=1×baselinemean;
CLART-dBM interpretation of HE Risk—Low.

### Clinical Outcome:

The patient was flagged with low risk of HE based on the CLART score derived using the methodology. The clinician reviewed the data and didn't take any action as the risk level was low.

### Case-5: Patient-9, Day 36:

### Data Captured Using the CLART Test:

CLART tests attempts=2 (1st test failed, 2nd test passed);
CLART Test completion time=1.2×baselinemean
CLART-dBM interpretation of HE Risk—Medium.

### Clinical Outcome:

The patient was flagged with medium risk of HE based on the CLART score derived using the methodology. The clinician reviewed the data and asked the patient to continue with the previously prescribed medication on day 32 (increased intake of lactulose);

### Case-6: Patient-9, Day 37:

### Data Captured Using the CLART Test:

CLART tests attempts=1 (1 completed successfully);
CLART Test completion time=2×baselinemean;
CLART-dBM interpretation of HE Risk=High.

### Clinical Outcome:

The patient was flagged with high risk of HE based on the CLART score derived using the methodology. The patient with the above score was admitted into the hospital with a diagnosis of HE secondary to poor diabetic control and dehydration. They were treated with fluids, insulin and adjustment of antidiabetic medication, staying in the hospital for 3 days. Endocrinology follow-up was arranged. The patient continued monitoring after returning home at had no liver-related events for the rest of their monitoring period.

### Case-7: Patient-9, Day 42:

### Data Captured Using the CLART Test:

CLART tests attempts=1 (completed successfully);
CLART Test completion time=1×baselinemean;
CLART-dBM interpretation of HE Risk—Low.

### Clinical Outcome:

The patient was flagged with low risk of HE based on the CLART score derived using the methodology. The clinician reviewed the data and didn't take any action as the risk level was low.

### Case-8: Patient-15, Day 31:

### Data Captured Using the CLART Test:

CLART tests attempts=1 (1 completed successfully);
CLART Test completion time=2.4×baselinemean;
CLART-dBM interpretation of HE Risk=High.

### Clinical Outcome:

The patient was flagged with high risk of HE based on the CLART score derived using the methodology. The patient was arranged for an urgent review by the Hepatology team at Accident and Emergency. At the hospital, he was found to be intoxicated but not encephalopathic, his blood tests were not significantly abnormal including only slightly elevated bilirubin of 27 mol/l, and he was discharged back home.

CLART digital biomarker is derived from combination of (a) percentage difference in time taken to complete the test and cumulative mean/median of completed tests and (b) failure count calculated based on the failed and completed tests.

CLART is a novel digital biomarker derived from assessment of cirrhosis patients using CyberLiver™ Animal Recognition Test (CLART) smartphone app and enables diagnosis of early hepatic encephalopathy remotely. It shows good comparability with other standard tests and high sensitivity for diagnostic performance in identifying HE. Given its ease-of-use, rapid testing (usually <30 s) and smartphone application, it provides a convenient tool for remote, sustainable management of cirrhosis.

The specific elements/steps and advantages of the invention are as follows:
it assesses a mental function of a patient as primary patient data;
it provides diagnosis of the development of hepatic encephalopathy in the cirrhotic patient;
it is a higher mental function test that uses Animal image recognition on a digital platform, as a key parameter to identify onset of early Hepatic Encephalopathy (HE);
the tests enable real-time monitoring of cognition and response to intervention;
it is a CLART digital biomarker (dBM) wherein the patient data are processed using a configurable algorithm that is based on evidence based research and clinical trials to diagnose Hepatic Encephalopathy in the cirrhotic patient;
it generates alerts to healthcare practitioners based on the diagnosis for early interventions.

Additional advantages of the system are as follows:
it can be integrated into Digital Therapeutic solutions for diagnosis and treatment of HE in decompensated Cirrhosis patients;
it enables a diagnosis of HE remotely, thereby alerting to the need for treatment and reducing re-admissions of patients with HE;
by avoiding the need for direct healthcare practitioner oversight of diagnostic tool output, the system is a low-cost, efficient, directly patient applied and reproducible method for continuous remote monitoring of HE, for patients with Cirrhosis;
it enables remote, real-time monitoring and clinical diagnostics, and personalized care within the safety of patients' homes. This achieves early direct clinical assessment, and early intervention when necessary, before the disease progresses, ideally avoiding preventable hospital admissions or/and reducing the severity of presentations by 15-35% in a preferred embodiment;
Animal recognition cognitive test is used in the CIT/CCPT instead of regular word/colour-based tests. The advantage of using animal recognition tests is to ensure that patients with colour blindness or other vision impairment can use the test and using animal recognition tests is simpler than using Animal Naming tests;
it enables remote access to patient care without the fear of hospital visits and cross infection and independent of patient and practitioner work schedules;
it is an easily accessible cognitive test through a simple smartphone device;
it generates actionable, real world insights that enable intelligent data-driven care management and clinical decision making based on evidence-based research through clinical trials;
it reduce practitioners' manual assessment time by using computer-based diagnosis;
it improves in everyday quality of patient life;
it enables early direct clinical reviews based on the data and analysis presented clearly to the practitioner;
it enables early and direct diagnosis to enable a treatment plan presented to patients to ensure immediate action to prevent HE progression;
it enables immediate alert systems to practitioners based on changes in patient data;
it enables reductions in mortality beyond standard of care.

1. A system for assessing hepatic encephalopathy of at least one patient, the system comprising:
patient application modules for patient devices, the patient application modules being configured to be used to take at least one animal recognition test for assessing hepatic encephalopathy, and to determine test results;
at least one interpretation module receiving the test results and configured to interpret the test results in order to determine parameters of hepatic encephalopathy,
at least one practitioner application module for at least one practitioner device the at least one practitioner module being configured to receive at least the parameters of hepatic encephalopathy to provide a diagnosis aid,
wherein the animal recognition test is based on animal images and corresponding text descriptions from a junior school repertoire, the text descriptions being names of animals to be selected; and
wherein the animal recognition test is made without colour shades parameters.

2. (canceled)

3. The system according to claim 1, wherein the animal recognition test is made by typing the right animal image or the right text description on a tactile screen.

4. The system according to claim 1, wherein the test results comprise at least:
a time to perform the test compared to a time baseline or median or mean;
a number of failed or successfully completed tests, and
a number of test attempts.

5. The system according to claim 4, wherein the test results comprise a score scaling a combination of the time to perform, the number of failed or successfully completed tests, and the number of attempts.

6. The system according to claim 1, wherein the parameters of hepatic encephalopathy comprise a positive/negative diagnosis, or a positive/negative probability.

7. The system according to claim 6, wherein the parameters of hepatic encephalopathy comprise a high, low or medium risk:
“low” meaning “low probability of developing a HE event”;
“medium” meaning a “high probability of developing a HE event”;
“High” meaning “detection of a HE event at the time of patient taking the test”.

8. The system according to claim 1, further comprising a prescription module to prescribe at least one new animal recognition test to be performed through the patient module of a patient.

9. The system according to claim 1, further comprising a reminder module configured to signal or remind the patient that a new animal recognition test is to be performed.

10. The system according to claim 1, further comprising at least one patient device comprising a corresponding patient module; at least one practitioner device comprising a corresponding practitioner module; and at least one server comprising the interpretation module.

11. (canceled)

12. A computer-implemented method for assessing hepatic encephalopathy of at least one patient, the method comprising:
a step for taking at least one animal recognition test to assess hepatic encephalopathy, and to determine test results;
a step for interpreting to interpret the test results, in order to determine parameters of hepatic encephalopathy,
a step for providing to provide a diagnosis aid based on the parameters of hepatic encephalopathy,
wherein the animal recognition test is based on animal images and corresponding text descriptions from a junior school repertoire, the text descriptions being names of animals to be selected; and
wherein the animal recognition test is made without colour shades parameters.

13. The method according to claim 12, wherein the animal recognition test is made by typing the right animal image or the right text description on a tactile screen.

14. The method according to claim 12, wherein the test results comprise at least:
a time to perform the test compared to a time baseline or median or mean;
a number of failed or successfully completed tests, and
a number of test attempts.

15. The method according to claim 14, wherein the parameters of hepatic encephalopathy comprise a high, low or medium risk:
“low” meaning “low probability of developing a HE event”;
“medium” meaning a “high probability of developing a HE event”;
“High” meaning “detection of a HE event at the time of a patient taking the test”.

16. The method according to claim 12, further comprising a step for prescribing at least one new animal recognition test to be performed by a patient.

17. The method according to claim 12, further comprising a step for reminding to signal or remind the patient that a new animal recognition test is to be performed.

18. The method according to claim 12, wherein:
the step for taking the at least one animal recognition test is made through at least one patient device;
the step for providing is made through at least one practitioner device; and
the step for interpreting is made through at least one server.

19. A computer program product comprising instructions which, when the program is executed by one or more computerised unit, causes the said computerised unit to carry out steps of a computer-implemented method for assessing hepatic encephalopathy of at least one patient, the method comprising:
a step for taking at least one animal recognition test to assess hepatic encephalopathy, and to determine test results;
a step for interpreting to interpret the test results, in order to determine parameters of hepatic encephalopathy,
a step for providing to provide a diagnosis aid based on the parameters of hepatic encephalopathy,
wherein the animal recognition test is based on animal images and corresponding text descriptions from a junior school repertoire, the text descriptions being names of animals to be selected; and
wherein the animal recognition test is made without colour shades parameters.
